(19) United States
(12) Patent Application Publication
(10) Pub. No.: US 2015/0335670 A1
(43) Pub. Date: Nov. 26, 2015

(54) Medical applications of Notoginsenoside Fc

(71) Applicants: Zhengtao WANG, (US); Bo GAO, (US); Li YANG, (US); Xiaojun WU, (US); Wei DOU, (US); Chun Yong HE, (US); Qing LIU, (US); Rufeng WANG, (US); Eryun ZHANG, (US); SHANGHAI UNIVERSITY OF TRADITIONAL CHINESE MEDICINE, Shanghai (CN)

(72) Inventors: Zhengtao Wang, Shanghai (CN); Bo Gao, Shanghai (CN); Li Yang, Shanghai (CN); Xiaojun Wu, Shanghai (CN); Wei Dou, Shanghai (CN); Chunyong He, Shanghai (CN); Qing Liu, Shanghai (CN); Rufeng Wang, Shanghai (CN); Eryun Zhang, Shanghai (CN)

(21) Appl. No.: 14/760,149

(22) PCT Filed: Jan. 9, 2014
(86) PCT No.: PCT/CN2014/070387

§ 371 Date: Jul. 9, 2015

(30) Foreign Application Priority Data
Jan. 9, 2013 (CN) 201310006881.3
Oct. 8, 2013 (CN) 201310465076.7
Nov. 25, 2013 (CN) 201310601145.2

(51) Int. Cl. A61K 31/704; A23L 1/30

(52) U.S. Cl. CPC A61K 31/704; A23L 1/30; A23V 2002/00

(57) ABSTRACT

A method for preparing a platelet aggregation inhibitor, a blood coagulation inhibitor, and a pharmaceutical composition or a food for preventing or treating thrombotic diseases, includes applying notoginsenoside Fc.

### CROSS REFERENCE OF RELATED APPLICATION

This is a U.S. National Stage under 35 U.S.C. 371 of the International Application PCT/CN2014/070387, filed Jan. 9, 2014, which claims priority under 35 U.S.C. 119(a-d) to CN 201310006881.3, filed Jan. 9, 2013; CN 201310465076.7, filed Oct. 8, 2013; and CN 201310601145.2, filed Nov. 25, 2013.

### BACKGROUND OF THE PRESENT INVENTION

1. Field of Invention

The present invention related to a medical field, and particularly to a medical application of notoginsenoside Fc.

2. Description of Related Arts

Thrombosis refers to the pathology process, in which some components of blood form emboli under special conditions in blood vessel (mostly capillary vessel), resulting in, the partially or fully blockage of blood vessels, thus ischemia in tissues or organs. The thrombogenesis involves in the aggregation of platelet, erythrocyte, and fibrin, which can occur within different arteries, veins and capillaries.

Thromboembolism refers to the pathological process, in which the thrombus falls off from the forming parts, blocks some blood vessels with the blood flow partially or fully, leading to the pathological process of corresponding tissue and organ, including ischemia, hypoxia, and necrosis (arterial thrombosis) and/or congestion edema (venous thrombosis).

In clinic, thrombotic disease contains all of the diseases provoked by both of the processes mentioned above. Due to its complexity, the etiology and pathogenesis of this disease have not been clarified yet. However, some studies in recent years showed that the occurrence and development of thrombotic diseases closely relate to the following two factors:

I) increase of abnormality, wherein many factors can cause the abnormal elevation of platelet number and activity, accordingly, increases the risky onset of thrombotic diseases. These factors include thrombocytosis or platelet destruction caused by mechanical, chemical, biological or immunological reactions. Conventionally, platelet factor is regarded as a key player in the pathogenesis of arterial thrombosis, such as myocardial infarction; and

II) increase of hematopexis, wherein blood clotting activity can be enhanced under many physiological and pathological conditions. For instance, stress responses caused by pregnancy, venerable age, wound infection, hyperlipidemia and malignant tumor can accelerate the rise of the levels and activities of coagulation factors. The hypercoagulable state of blood is the basis for the pathogenesis of thrombotic diseases.

Myocardial ischemia refers to the reduction of the heart blood perfusion, a pathological state which can lead to the shortage of cardiac oxygen supply, thus the abnormal condition of myocardial energy metabolism. Coronary artery stenosis or occlusion caused by coronary artery atherosclerosis is the leading and the most common cause of myocardial ischemia, which may further give rise to myocardial hypoxia, and the heart disease that is often known as “coronary heart disease”.

Myocardial infarction is a partial necrosis of the heart muscle caused by severe persistent ischemia due to coronary occlusion and blood flow interruption, which is accompanied with intense and persistent retrosternal pain, fever, increased leucocytes, accelerated erythrocyte sedimentation rate, elevated serum myocardial enzyme activity and progressive change of electrocardiogram, leading to final cardiac arrhythmia, shock or heart failure.

Notoginsenoside Fc is a natural saponin molecule that is mainly present in Panax notoginseng (Burk.) F. H. Chen. Notoginsenosides have been shown to exert multiple functions in lowering hyperlipidemia, anti-tumor, anti-oxidation, etc.

### SUMMARY OF THE PRESENT INVENTION

An object of the present invention is to provide a new medical application of notoginsenoside Fc.

Specifically, in one aspect, the present invention provides a method for preparing a platelet aggregation inhibitor, comprising applying notoginsenoside Fc.

Preferably, a concentration of the notoginsenoside Fc is ranged from 0.01 μM to 400 μM.

In another aspect, the present invention provides a method for preparing a blood coagulation inhibitor, comprising applying notoginsenoside Fc.

Preferably, a concentration of the notoginsenoside Fc is ranged from 0.01 mg/ml to 240 mg/ml.

In another aspect, the present invention provides a method for preparing a pharmaceutical composition or a food for preventing or treating a thrombotic disease, comprising applying notoginsenoside Fc.

Preferably, a concentration of the notoginsenoside Fc is ranged from 0.01 mg/kg to 240 mg/kg.

Preferably, the thrombotic disease is mainly caused by platelet hyperfunction or increased blood coagulation.

In another aspect, the present invention provides a method for preparing a pharmaceutical composition for preventing or treating a thrombotic disease, comprising applying notoginsenoside Fc, wherein the thrombotic disease comprises arterial thrombosis, venous thrombosis and capillary thrombosis; a concentration of the notoginsenoside Fc is ranged from 0.01 mg/kg to 240 mg/kg.

In another aspect, the present invention provides a pharmaceutical composition or a food for preventing or treating a thrombotic disease, comprising a therapeutically effective amount of notoginsenoside Fc.

Preferably, the thrombotic disease is caused by platelet hyperfunction or increased blood coagulation.

In another aspect, the present invention provides a method for preparing a pharmaceutical composition or a food for preventing or treating myocardial ischemia, comprising applying notoginsenoside Fc.

In another aspect, the present invention provides a method for preparing a pharmaceutical composition or a food for preventing or treating myocardial infarction, comprising applying notoginsenoside Fc.

In another aspect, the present invention provides a pharmaceutical composition or a food for preventing or treating myocardial ischemia, comprising a therapeutically effective amount of notoginsenoside Fc.

In another aspect, the present invention provides a pharmaceutical composition or a food for preventing or treating myocardial infarction, comprising a therapeutically effective amount of notoginsenoside Fc.

These and other objectives, features, and advantages of the present invention will become apparent from the following detailed description, the accompanying drawings, and the appended claims.

### BRIEF DESCRIPTION OF THE DRAWINGS

FIG. 1 illustrates an effect of notoginsenoside Fc on a platelet aggregation rate.

FIG. 2 illustrates a dose-response relationship of the notoginsenoside Fc on the platelet aggregation rate.

FIG. 3 illustrates an effect of the notoginsenoside Fc on the platelet aggregation rate of an animal.

FIG. 4 illustrates an effect of the notoginsenoside Fc on a bleeding time of rat tails.

FIG. 5 illustrates an effect of the notoginsenoside Fc on an activated partial thromboplatin time of rat plasma.

FIG. 6 illustrates an effect of the notoginsenoside Fc on four thromboplatin indexes of rats and of humans.

FIG. 7 illustrates an effect of the notoginsenoside Fc on a prothrombin time of humans.

FIG. 8 illustrates an effect of the notoginsenoside Fc on an area percentage of acute myocardial infarction in rats.

FIG. 9 is a pathohistological image of a rat myocardial infarction surgery control group.

FIG. 10 is a pathohistological image of a rat myocardial infarction surgery sham group.

FIG. 11 is a pathohistological image of a rat myocardial infarction positive medicine group (with a dose of 4.1 mg/kg).

FIG. 12 illustrates a pathohistological image of a rat myocardial infarction medicine-treated group (with a dose of 4 mg/kg).

FIG. 13 illustrates a pathohistological image of a rat myocardial infarction medicine-treated group (with a dose of 12 mg/kg).

### DETAILED DESCRIPTION OF THE PREFERRED EMBODIMENT

The advent of the present invention is based on an unexpected discovery, showing that notoginsenoside Fc could inhibit platelet aggregation, prolong activated partial thromboplatin time (APTT), and significantly reduce the percentage of the area of myocardial infarction in rats. Therefore, notoginsenoside Fc can be developed to inhibit platelet aggregation as a platelet aggregation inhibitor or as a coagulation inhibitor. As known to a professional in the field, the “platelet aggregation inhibitor” and “coagulation inhibitor” may be in various forms, including, but not limited to drugs, healthy products, food, article of everyday use, and so on. “Platelet aggregation inhibitor” and “coagulation inhibitor” prepared by notoginsenoside Fc can be used to prevent or treat various thrombotic diseases caused by the platelet hyperfunction or increased blood coagulation. These include but not limit to: arterial thrombosis, venous thrombosis and capillary thrombosis, myocardial ischemia, myocardial infarction, and so on.

Therefore, the present invention provides a method for preparing a platelet aggregation inhibitor, comprising applying notoginsenoside Fc.

Preferably, a concentration of the notoginsenoside Fc is ranged from 0.01 μM to 400 μM.

The present invention also provides a method for preparing a blood coagulation inhibitor, comprising applying notoginsenoside Fc.

Preferably, a concentration of the notoginsenoside Fc is ranged from 0.01 mg/ml to 240 mg/ml.

The present invention also provides a method for preparing a pharmaceutical composition or a food for preventing or treating a thrombotic disease, comprising applying notoginsenoside Fc.

Preferably, a concentration of the notoginsenoside Fc is ranged from 0.01 mg/kg to 240 mg/kg.

Preferably, the thrombotic disease is mainly caused by platelet hyperfunction or increased blood coagulation.

The present invention also provides a method for preparing a pharmaceutical composition for preventing or treating a thrombotic disease, comprising applying notoginsenoside Fc, wherein the thrombotic disease comprises arterial thrombosis, venous thrombosis and capillary thrombosis; a concentration of the notoginsenoside Fc is ranged from 0.01 mg/kg to 240 mg/kg.

The present invention also provides a pharmaceutical composition or a food for preventing or treating a thrombotic disease, comprising a therapeutically effective amount of notoginsenoside Fc.

Preferably, the thrombotic disease is caused by platelet hyperfunction or increased blood coagulation.

The present invention also provides a method for preparing a pharmaceutical composition or a food for preventing or treating myocardial ischemia, comprising applying notoginsenoside Fc.

The present invention also provides a method for preparing a pharmaceutical composition or a food for preventing or treating myocardial infarction, comprising applying notoginsenoside Fc.

The present invention also provides a pharmaceutical composition or a food for preventing or treating myocardial ischemia, comprising a therapeutically effective amount of notoginsenoside Fc.

The present invention also provides a pharmaceutical composition or a food for preventing or treating myocardial infarction, comprising a therapeutically effective amount of notoginsenoside Fc.

In the present invention, a molecular formula of the notoginsenoside Fc is: C₅₈H₉₈O₂₆, and a molecular weight thereof is 1210.63. A structural formula thereof is as follows.

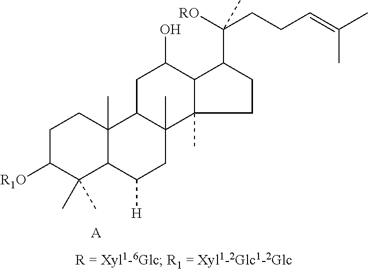

The notoginsenoside Fc in the present invention is commercially available from Sigma Chemical Company, Chengdu Munster Biotechnology Co., Ltd. or isolated from leaves of Panax Notoginseng by conventional methods in the field. A purity thereof complies with pharmaceutical standards.

As an instance, the notoginsenoside Fc of the present invention may be used alone or be used in a form of a pharmaceutical composition. The pharmaceutical composition comprises the notoginsenoside Fc as an active ingredient, and a pharmaceutical carrier. Preferably, the pharmaceutical composition of the present invention comprises 0.1% to 99.9% (w/w) notoginsenoside Fc. The pharmaceutical carrier will not undermine pharmaceutical activity of the notoginsenoside Fc and an effective dosage, which means when the pharmaceutical carrier is effective, the dosage is not toxic to humans.

The pharmaceutical carrier comprises lecithin, aluminum stearate, alumina, ion exchange material, self-emulsifying drug delivery system, tween or other surface active agents, serum proteins, buffer substances such as phosphates, amino acetic acid, sorbic acid, water and salts, and electrolytes such as protamine sulfate, disodium hydrogen phosphate, potassium hydrogen phosphate, sodium chloride, zinc salts, magnesium silicate, and partial glyceride mixtures of saturated fatty acid.

Other common pharmaceutical adjuvants comprise binder (microcrystalline cellulose), fillers (starch, glucose, anhydrous lactose and lactose beads), disintegrating agents (cross-linked PVP, cross-linked sodium carboxymethyl starch, cross-linked sodium carboxymethylcellulose, low substituted hydroxypropylcellulose), lubricants (magnesium stearate), absorption accelerators, adsorption carrier, flavoring agents, sweeteners, excipients, diluents, and wetting agents.

The notoginsenoside Fc and the pharmaceutical compositions of the present invention can be prepared by conventional methods in the field and can be administered by enteral or parenteral or topical route. An oral preparation comprises capsules, tablets, oral liquid, granules, pills, powder and pastes; a parenteral preparation comprises injection liquid; a topical preparation comprises creams, patches, ointments and sprays. The oral preparation is preferred.

The administration routes of the notoginsenoside Fc and the pharmaceutical compositions of the present invention may be oral, sublingual, transdermal, intramuscular, or subcutaneous, mucocutaneous, vein, urethra, and vagina.

Furthermore, antioxidants, pigments, enzymes and other food additives may be added into the notoginsenoside Fc in the present invention for preparing healthy foods according to conventional methods in the field.

The present invention is further elucidated with embodiments below. It should be understood that these embodiments are merely illustration of the present invention, and are not intended to limit the scope of the present invention. The experimental conditions in the following embodiments are generally in accordance with conventional or recommended approaches by the manufacturers unless specifically mentioned. All percentages, ratios, proportions, or parts are measured by weight unless otherwise indicated.

Unless otherwise defined, all terminologies and scientific terms used in this text have the same meaning that can be easily understood by the professionals in the field. In addition, any method and material similar or equal with the recorded content can be applied to the methods of the present invention. The methods and materials of the embodiments described herein are for the purpose of demonstration only.

Aforementioned features in the present invention, or embodiments can be arbitrarily combined. All of the features disclosed in the descriptions of this patent can be used in different combinations and substituted by the same, equal, or similar alternatives. Therefore, unless especially noted, the disclosed features are generally regarded as equal or similar examples.

### Embodiment 1

Notoginsenoside Fc used in the embodiment 1 was isolated and prepared by Institute of Chinese Materia Medica, Shanghai University of Traditional Chinese Medicine. Clopidogrel, a positive control drug, was purchased from China National Institutes for Food and Drug Control. Purity of both chemicals meets medical standards.

1 Materials

1.1 Reagents

Notoginsenoside Fc (molecular weight 1211.38, HPLC purity≧99%, from Institute of Chinese Materia Medica), clopidogrel (molecular weight 419.90, HPLC purity≧98%, from National Institutes for Drug Control), standard human plasma, reagents for measurement of four indexes of blood coagulation (from Siemens, Germany), and adenosine diphosphate (ADP) (from Sigma, USA).

1.2 Animals

Male Sprague-Dawley rats weighing 230±20 g were provided by Experimental Animal Center of Shanghai University of Traditional Chinese Medicine with a certificate of conformity as SYXK (Shanghai) 2008-0016. All animals were kept under normal conditions with free access to food and water.

2 Methods

2.1 Effect of Notoginsenoside Fc on Platelet Aggregation

2.1.1 In vitro platelet aggregation assay: blood samples were placed in a centrifuge tube pre-coated with sodium citrate and centrifuged at 100 g for 5 min. The supernatant was collected and designated as rich platelet (PRP). After addition of prostacyclin (2 μg/ml), PRP was centrifuged at 700 g for 5 min and washed twice with 0.9% saline. The washed platelets were re-suspended in Tyrode buffer and diluted at a density of 4×10⁸ cells/ml for further utilization. Platelet aggregation assay was provided according to a nephelometry method: platelet plasma was placed in cuvette; after addition of a inducing agent (ADP: 5-adenosine diphosphate disodium salt: 1×10⁻⁴ mol/L), the platelet plasma was stirred with magnetic particles, wherein platelet was gradually aggregated, plasma turbidity was reduced, and transmittance was increased. Changes were recorded for drawing a dynamic curve of platelet aggregation. Aggregation rate and transmittance for PRP were regarded as 0; aggregation rate and transmittance of suspension were regarded as 100%. A platelet aggregation instrument was used for automatic measurement, recording, and drawing the platelet aggregation curve. For judging inhibiting or promoting of aggregation, it was mainly depending on whether synergy or antagonism happened with the inducing agent; wherein synergy was to promote platelet aggregation, antagonism was to inhibit platelet aggregation.

2.1.2 Whole animal thrombogenesis assay: when arterial blood platelets contact a rough surface of a wire, the platelets are adhered to the wire and form platelet thrombus on a surface thereof. When platelet adhesion and aggregation function is suppressed, thrombus is light. Therefore, thrombus weight can measure platelet adhesion and aggregation functions.

2.1.3 Effect of Notoginsenoside Fc on Blood Coagulation

2.1.3.1 Bleeding time assay: rat tail was transected 0.5 cm from the tip to induce bleeding. And the bleeding time was defined as the time from the start of transection to bleeding cessation. The time at which the flow of blood ceased for 30 s was considered as bleeding cessation.

2.1.3.2 Four indexes of blood coagulation: i) Prothrombin time (PT) mainly reflects extrinsic coagulation conditions. Its value is increased when the levels of coagulation factor IX and XI in plasma are reduced, which usually occurs in hemophilia, hemophilia B and factor XI deficiency patients. Conversely, the reduction of it is seen in hypercoagulable state, when the procoagulant substances get into blood and the activity of coagulation factors is increased. ii) Activated partial thromboplastin time (APTT) reflects mainly intrinsic coagulation conditions. Its value is changed under the similar condition as PT. iii) Thrombin time (TT) mainly reflects the abnormality in the conversion of fibrinogen into fibrin. Its value is elevated in DIC hyperfibrinolysis period, afibrinogenemia and hemoglobin hyperlipidemia, and when degradation products (FDPs) of blood fibrin are increased. iv) Fibrinogen (Fibrinogen, FIB) mainly reflects the content of fibrinogen in plasma. Generally, its concentration is elevated in acute myocardial infarction. The reduction of it is observed in DIC dissolution of low consumption of coagulation, primary fibrinolysis, severe hepatitis, hepatic cirrhosis.

2.2 Grouping and Administration Methods

Blank control group: 0.9% physiological saline (NS).

Model group: adenosine diphosphate (ADP) (10 mM).

Positive control group: clopidogrel (5 mM).

Treatment groups: six rats for each group, Fc dissolved in double distilled water (prepared with different concentrations).

2.3 Statistical Analysis

All experiments were repeated at least three times. The data were expressed as mean±standard deviations (SD). Difference among groups was analyzed using one-way analysis of variance (One-way ANOVA) followed by LSD test with SPSS 13.0 software. The value of P<0.05 was considered statistically significant.

3. Results

3.1 Notoginsenoside Fc Inhibits Platelet Aggregation

3.1.1 Effect of Fc on Platelet Aggregation Rate

FIG. 1 and table 1 reflected the influence of notoginsenoside Fc on platelet aggregation rate. As shown in FIG. 1, ADP alone induced 52.9% platelet to aggregate. When Fc (200 μM) was added in, the aggregation rate was reduced to 25.8%, which was significantly lower than that in both model group (*P<0.05) and positive group (*P<0.05). The result illustrated that notoginsenoside Fc inhibits platelet aggregation.

Furthermore, referring to FIG. 1, aggregation rate of total extract group was 40.1%, which was significantly higher than the Fc treatment group (25.8%) (**P<0.01), indicating that the notoginsenoside Fc monomer has better efficacy to inhibit platelet aggregation than the total extract group.

TABLE 1
Dose-response relationship of notoginsenoside Fc on
platelet aggregation rate

| Dose (μM) | aggregation rate (%) | SD |
|---|---|---|
| 0.1 | 47.98 | 3.56 |
| 10 | 42.83 | 2.55 |
| 20 | 32.33 | 3.51 |
| 30 | 31.60 | 4.86 |
| 40 | 28.13 | 3.77 |

3.1.2 Dose-Response Relationship of Fc on Platelet Aggregation Rate

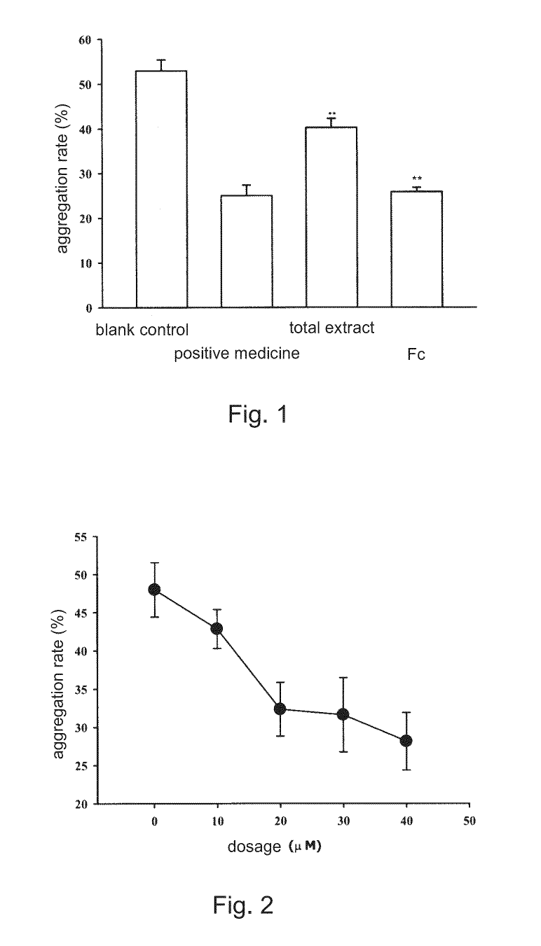

FIG. 2 illustrated a dose-response relationship of the notoginsenoside Fc on the platelet aggregation rate. Referring to FIG. 2, the dose-response relationship of the Fc (0.1 μM, 10 μM, 20 μM, 30 μM and 40 μM) on inhibition of platelet aggregation is illustrated, wherein from 0.1 μM to 40 μM, platelet aggregation rate was decreased. The platelet aggregation rate was decreased with the increase of Fc concentration, wherein the aggregation rate was decreased from 47.9% to 28.1%, a decreasing amplitude thereof was up to 19.8%. And the IC₅₀ of Fc was calculated as 13.5 μM.

3.1.3 Effect of Fc on Platelet Aggregation In Vivo

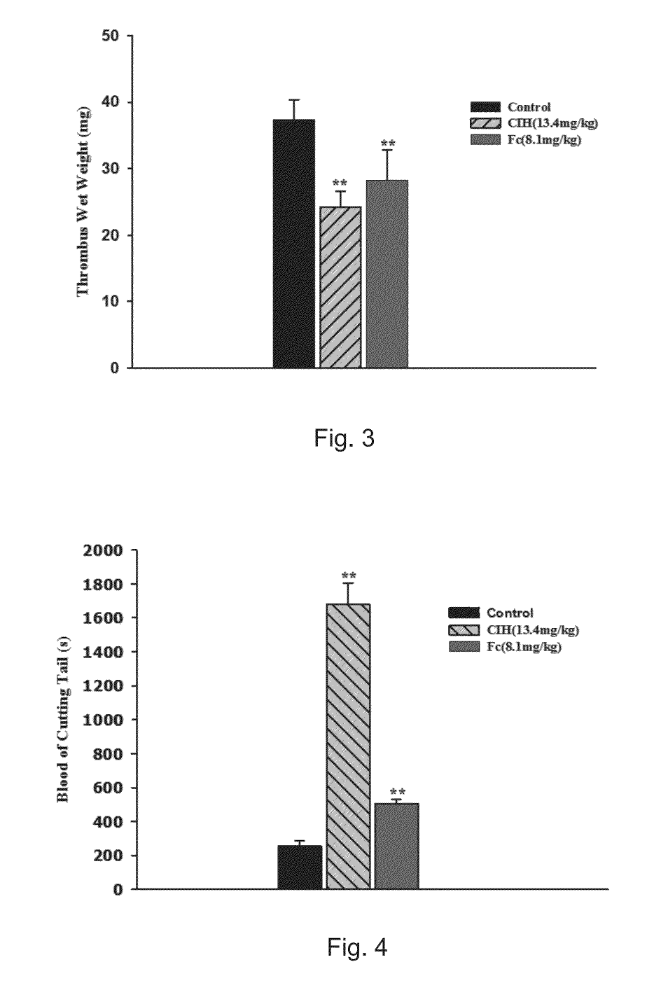

FIG. 3 showed that Fc influenced the platelet aggregation in vivo. Referring to FIG. 3, effect of the notoginsenoside Fc on the platelet aggregation function is illustrated. By weighing the wet weight of thrombus, we found that Fc could actively reduce thrombogenesis in vivo. In the extracorporeal circulation model, the rats were intravenous administered with Fc at dose of 8.1 mg/kg. Five minutes later, the extracorporeal circulation was initiated and stopped after fifteen minutes. The thrombus formed on the thread was weighed. Significant difference was found between the blank control group and the Fc treatment group (**P<0.01).

3.2 Notoginsenoside Fc Prevents Blood Coagulation

3.2.1 Effect of Fc on Bleeding Time

FIG. 4 reflected the effect of Fc on rat tail bleeding time. Referring to FIG. 4, Fc significantly prolonged rat tail bleeding time. A weight of the FC thrombus was 28.2 mg, which was significantly less than 37.2 mg of the blank control group (**P<0.01).

3.3 Effect of Fc on Four Indexes of Blood Coagulation

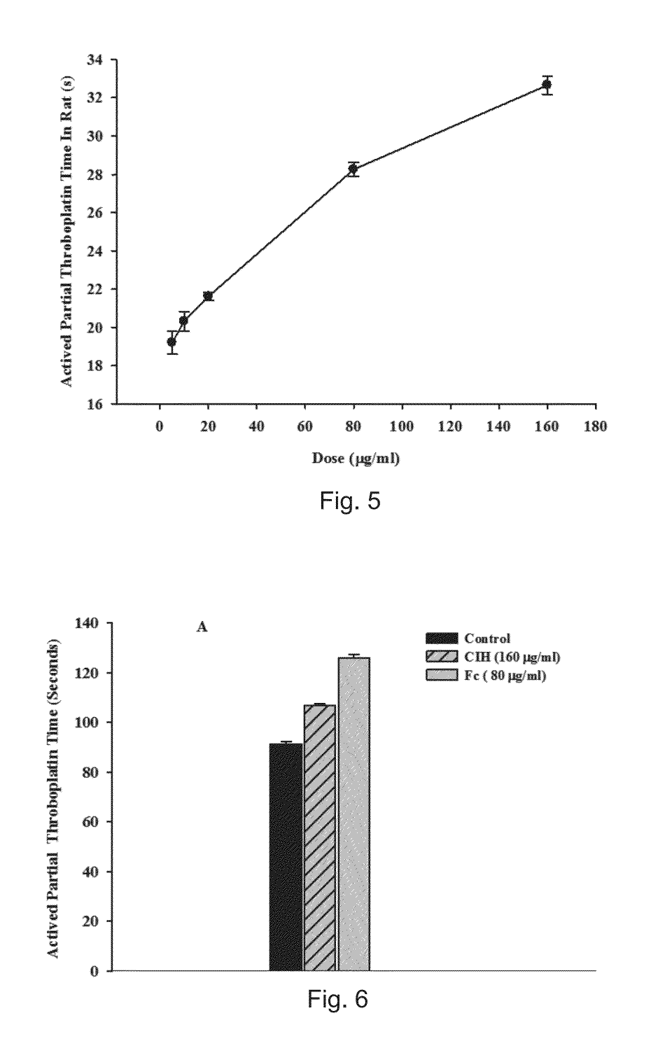

FIG. 5 and table 2 reflected the effect of Fc on rat activated partial thromboplastin time (APTT). Referring to FIG. 5, when used from 5 μg/ml, 10 μg/ml, 20 μg/ml, 80 μg/ml and 160 μg/ml, Fc could dose-dependently prolong APTT. As the concentration of Fc increased from 5 μg/ml to 160 μg/ml, APTT was prolonged from 19 seconds to 32 seconds, wherein a difference was 13 seconds.

TABLE 2
Notoginsenoside Fc dose-dependently prolonged rat APTT

| APTT time (μg/ml) | Time (sec) | SD |
|---|---|---|
| 5.0000 | 19.20 | 0.60 |
| 10.0000 | 20.31 | 0.51 |
| 20.0000 | 21.61 | 0.22 |
| 80.0000 | 28.25 | 0.37 |
| 160.0000 | 32.65 | 0.47 |

FIG. 6 reflected the effect of Fc on the four indexes of human blood coagulation. Referring to FIG. 6, the Fc significantly prolonged APTT. At 80 μg/ml, APTT was up to 126 s, which was significantly longer than the time (91 s) of the blank control group (**P<0.01). PT was also longer (*P<0.05). When Ftl was at 240 μg/ml, APTT was only 82 s, which was significantly longer than the time (91 s) of the blank control group (**P<0.01). PT was only 17 s, which was significantly shorter than the time (20 s) of the blank control group (*P<0.05).

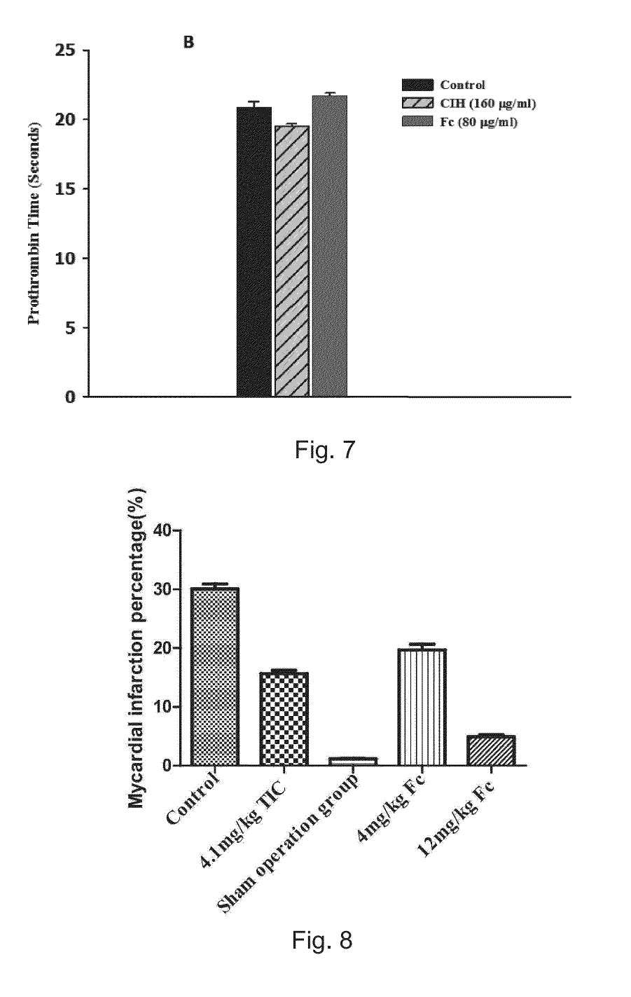

FIG. 7 reflected the effect of Fc on human PT. Fc could extend PT remarkably than the control (*P<0.05). PT was only 17 s, which was significantly shorter than the time (20 s) of the blank control group (*P<0.05).

### Example 2

### Notoginsenoside Fc Reduced Infarct Size and Alleviated Myocardial Ischemia in Rats

1 Materials

1.1 Reagents

Notoginsenoside Fc (molecular weight 1211.38, HPLC purity≧99%, from Institute of Chinese Materia Medica), and clopidogrel (molecular weight 419.90, HPLC purity≧98%, from National Institutes for Drug Control).

1.2 Animals

Male Sprague-Dawley rats weighing 230±20 g were provided by Experimental Animal Center of Shanghai University of Traditional Chinese Medicine with the certificate of conformity as SYXK (Shanghai) 2008-0016. All animals were kept under normal conditions with free access to food and water.

2. Methods

Model preparing method: 30 SD rats (Shanghai University of Traditional Chinese Medicine Experimental Animal Center), weight: 320-370 g; rat was treated with anesthesia with pentobarbital (35 mg/kg), supine fixing; neck midline incision, separating trachea, tracheotomy, inserting a breathing tube, adjusting a frequency and tidal volume of a ventilator; median sternotomy thoracotomy (3-4 intercostal), using thin surgical needle and line six for starting at a left atrial appendage, and ligation at pulmonary cone. Successful standard thereof was that: observing with naked eyes (anterior descending artery blood supply area was darken or pale). Chest was quickly closed. For the sham group, the line passed the left anterior descending coronary artery bypass without ligation.

TTC staining: rat hearts were frozen at −20° C., and were taken out after being hard; the rat hearts were left-coronally excised into 5-6 pieces at a thickness of 3-4 mm. Then the rat hearts were placed in 0.5% TTC solution, and incubated at 37° C. for 15 min. The pieces were taken out and fixed in 4% formaldehyde solution.

Collection indicator: heart pieces showed two different colors, red for normal cardiac tissue; white or gray-black for necrotic myocardium. The hearts were cut according to two different colors and drained water with filter paper before weighing. Calculation formula was as follows: ischemic area (%)=total weight÷infarcted heart weight×100%. Meanwhile, the hearts were partly acquired for pathological analysis.

3 Experimental Design

Experimental design for acute myocardial infarction

Control group: six rats, administered with 0.9% physiological saline (NS) after surgery.

Sham operation group: six rats, opening chest and passing line without ligation.

Positive medicine group: six rats, administered with ticagrelor (4.1 mg/kg) after surgery.

4 mg/kg Fc treatment group: six rats, administered with Fc (4 mg/kg) after surgery.

12 mg/kg Fc treatment group: six rats, administered with Fc (12 mg/kg) after surgery.

4. Results (as Shown in Table 3)

FIG. 8 showed that the Fc significantly reduced the percentage of the infarcted area. Furthermore, dose-response relationship was significant between the 4 mg/kg Fc treatment group and 12 mg/kg Fc treatment group (P<0.001). Moreover, 12 mg/kg Fc treatment group reduced more myocardial infarction than the positive medicine group (4.1 mg/kg) (P<0.01).

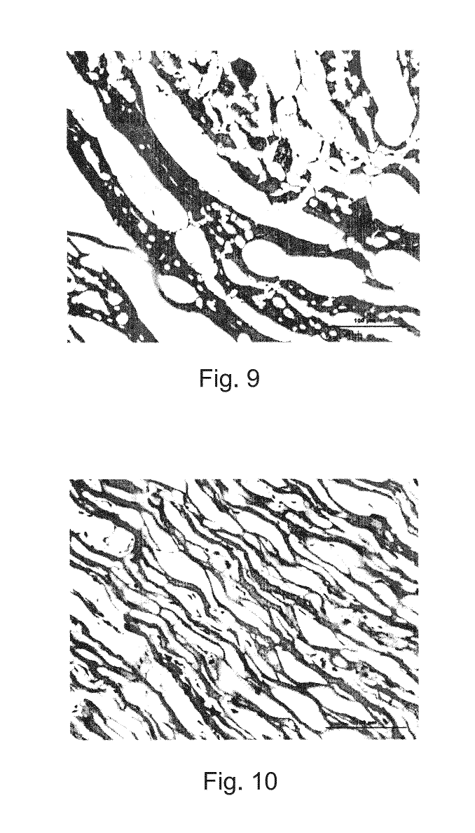

Referring to FIG. 9, myocytes in control group rats were disarrayed with eosinophilic cytoplasm stained with concentrated dye. Cytoplasm of partial cells was reduced, while that of other partial cells was increased. Moreover, the nuclei of the myocytes were unevenly distributed. In local area, multinucleated cells could be found. Vascular degeneration was visible at intercellular matrix. However, inflammatory cell was rare.

Referring to FIG. 10, the arrangement of myocardial cells in sham operation group rats was normal. Cytoplasm of the cells was eosinophilic. Meanwhile, cell nuclei were uniform in size and distributed evenly. No inflammatory cells could be found at intercellular matrix.

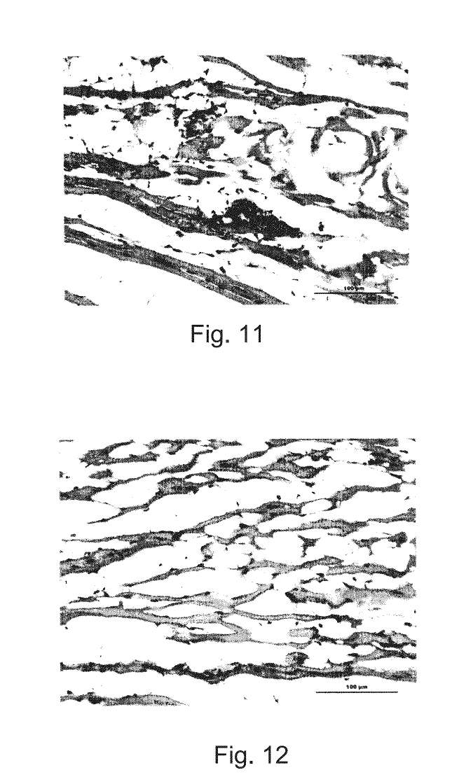

Referring to FIG. 11, after TIC treatment, the arrangement of myocardial cells was disordered in local area. Cytoplasm of the cells was eosinophilic. Cell nuclei were uniform in size but distributed unevenly. Multinucleated cells could be found as well as vascular degeneration. But inflammatory cell was rare within intercellular matrix.

Referring to FIG. 12, when treated with Fc at 4 mg/kg, the arrangement of most of the myocardial cells was normal. Cytoplasm of the cells was eosinophilic. But cell nuclei gathered in local location. Infiltration of a few inflammatory cells was visible at intercellular matrix as well as vascular degeneration.

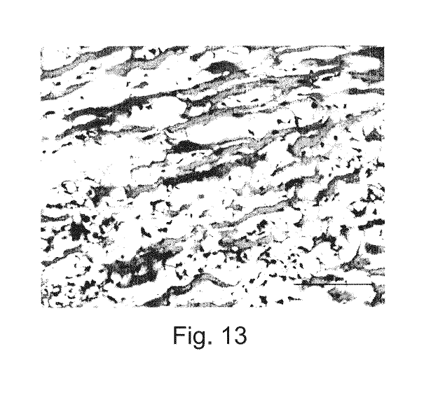

Referring to FIG. 13, in cardiac tissues treated with Fc at 12 mg/kg, the arrangement of the myocardial cells was almost normal. But in local area, cell cytoplasm was dispersed. In general, cell cytoplasm was abundant. The cell nuclei showed increased size. The vascular degeneration as well as infiltration of inflammatory cells was alleviated.

TABLE 3

| Surgical control group | | | | |
|---|---|---|---|---|
| (Surgical Myocardial | Positive group | Sham | 4 mg/kg | 12 mg/kg |
| infarction area) | (TIC, ticagrelor) | group | group Fc | group Fc |
| 27.13321607 | 13.85011632 | 1.39306267 | 22.79706066 | 5.463254414 |
| 31.41280862 | 14.23524998 | 0.99470543 | 19.04818289 | 4.891520805 |
| 30.93575991 | 16.68880547 | 1.002895263 | 19.74481346 | 5.189625308 |
| 28.6896878 | 16.94221624 | 0.881343219 | 16.87848121 | 4.595102825 |
| 29.73497402 | 15.15228858 | 1.091739174 | 17.27245359 | 5.842934044 |
| 32.6369087 | 16.99771291 | 1.464660163 | 22.30639098 | 3.482079574 |

Most aspects of the present invention have been elucidated as above. However, it should be understood that, without departing from the spirit of the present invention, professionals in the field may change or modify the compound with equivalent efficacy. Such changes and modifications will also fall within the coverage of the claims of current patent.

What is claimed is:

1. A method for preparing a platelet aggregation inhibitor, comprising applying notoginsenoside Fc.

2. The method, as recited in claim 1, wherein a concentration of the notoginsenoside Fc is ranged from 0.01 μM to 400 μM.

3. A method for preparing a blood coagulation inhibitor, comprising applying notoginsenoside Fc.

4. The method, as recited in claim 3, wherein a concentration of the notoginsenoside Fc is ranged from 0.01 mg/ml to 240 mg/ml.

5. A method for preparing a pharmaceutical composition or a food for preventing or treating a thrombotic disease, comprising applying notoginsenoside Fc.

6. The method, as recited in claim 5, wherein a concentration of the notoginsenoside Fc is ranged from 0.01 mg/kg to 240 mg/kg.

7. The method, as recited in claim 5, wherein the thrombotic disease is caused by platelet hyperfunction or increased blood coagulation.

8. A method for preparing a pharmaceutical composition for preventing or treating a thrombotic disease, comprising applying notoginsenoside Fc, wherein the thrombotic disease comprises arterial thrombosis, venous thrombosis and capillary thrombosis; a concentration of the notoginsenoside Fc is ranged from 0.01 mg/kg to 240 mg/kg.

9. A pharmaceutical composition or a food for preventing or treating a thrombotic disease, comprising a therapeutically effective amount of notoginsenoside Fc.

10. The pharmaceutical composition or the food, as recited in claim 9, wherein the thrombotic disease is caused by platelet hyperfunction or increased blood coagulation.

11. A method for preparing a pharmaceutical composition or a food for preventing or treating myocardial ischemia, comprising applying notoginsenoside Fc.

12. A method for preparing a pharmaceutical composition or a food for preventing or treating myocardial infarction, comprising applying notoginsenoside Fc.

13. A pharmaceutical composition or a food for preventing or treating myocardial ischemia, comprising a therapeutically effective amount of notoginsenoside Fc.

14. A pharmaceutical composition or a food for preventing or treating myocardial infarction, comprising a therapeutically effective amount of notoginsenoside Fc.
